(19) United States
(12) Plant Patent Application Publication
(10) Pub. No.: US 2005/0273892 P1
(43) Pub. Date: Dec. 8, 2005

(54) 'Pendleton Cherry' cultivar

(50) PRUNUS AVIUM; CHERRY

(76) Inventors: Roger D. Way, Stanley, NY (US); Robert L. Andersen, Geneva, NY (US); Susan K. Brown, Geneva, NY (US)

Correspondence Address: International Plant Management, Inc., 55826 60th Avenue, Lawrence, MI, 49064, US

(21) Appl. No.: 10/863,179

(22) Filed: Jun. 8, 2004

(51) Int. Cl. A01H 5/00

(52) U.S. Cl. PLT/181

(57) ABSTRACT

A new and distinct variety of sweet cherry tree, ‘Pendleton Cherry’, originating as the result of a cross between ‘Yellow Glass’, an unpatented selection, and ‘Emperor Francis’, an unpatented selection. This new variety is unique because its tree has proven to be reliable to produce regular, heavy crops of mid-late season, blushed skin, yellow fleshed fruit that are suitable for commercial uses in processing markets in Eastern states of North America.

### FIELD OF THE INVENTION

A new and distinct cultivar of sweet cherry tree originated as a seedling of Prunus avium in the breeding program of Cornell University and is hereinafter referred to as ‘Pendleton’ sweet cherry. This new variety is unique because its tree has proven to be reliable to produce regular, heavy crops of mid-late season, blushed skin, yellow fleshed fruit that are suitable for commercial uses in processing markets in Eastern states of North America. It possesses another unique feature in that it will pollinate both ‘Emperor Francis’ and ‘Ulster’, the two most important mid-season processing sweet cherry varieties in the Eastern United States sweet cherry processing industry. Here to for, no effective pollinator variety has been available for this purpose, which could be relied upon to also yield heavily itself.

### SUMMARY OF THE INVENTION

This new and distinct variety of sweet cherry was discovered by Dr. Roger Way (retired), a plant breeder at the New York State Agricultural Experiment Station, Geneva, N.Y., a research unit of Cornell University, hereinafter referred to as Geneva Experiment Station. By breeding methodology convention at the Geneva Experiment Station it was designated NY 8182. This selection was initially assigned Experiment Station Breeding Record Number 62231, a hybrid population of trees that resulted from hybridizing the varieties ‘Yellow Glass’×‘Emperor Francis’ in 1962 and this seedling was designated as NY 8182 when it was planted in 1964 as part of a population of 85 siblings possessing the same parentage. The orchard location where the seedling was grown and first noticed was designated as Orchard Number 26, Row 9, Tree 122. This seedling was selected because in 1969 Cornell scientists purposely looked for and chose seedlings with heavy cropping characteristics for the relatively unfavorable weather conditions existing for sweet cherry production at Geneva, N.Y., where both cold winters and spring frosts frequently cause serious damage to trees and flower buds of many varieties of sweet cherries. Cherry varieties for processing uses that would ripen between ‘Emperor Francis’ and ‘Yellow Gold’ were also sought. This seedling also was chosen due to its ripening time that fits between its two parents. NY 1725 was propagated on Mazzard seedling rootstocks in 1969 so as to create more trees for further evaluations on the Geneva Experiment Station. Subsequently it has been propagated on Mahaleb seedling and ‘Gisela 5’ rootstocks remains true to the description herein.

### A BRIEF DESCRIPTION OF THE DRAWINGS

The accompanying photographs show typical specimens of the new variety as depicted in color as nearly true as is reasonably possible in color illustrations of this character. These specimens were obtained at the Geneva Experiment Station, Geneva, N.Y.

FIG. 1. illustrates a bowl containing several intact fruit at maturity with the stem characteristics accurately depicted.

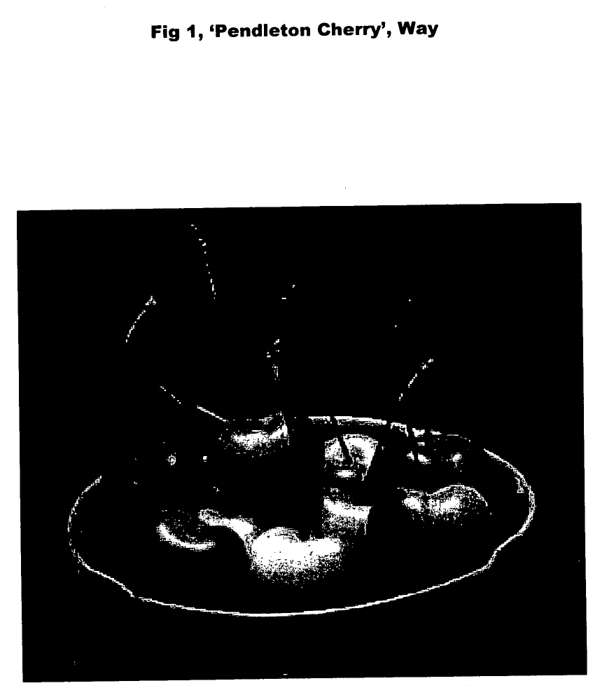

### A BOTANICAL DESCRIPTION OF THE PLANT

A detailed description of the ‘Pendleton cherry’ cultivar follows using The Royal Society of London Colour identification except where general color terms are sufficient.
Parentage:
   A hybrid seedling of the cross.—‘Yellow Glass’×‘Emperor Francis’.

Locality of the original discovery and observations is Orchard Number 26, Row 9, Tree 122 (as assigned the designation NY 8182), Geneva Experiment Station, Geneva, N.Y., U.S.A.
Tree:
   Age and rootstock of specimen.—6 years old on ‘Gisela 5’ rootstock.
   Height.—2.0 M.
   Width.—1.5 M.
   Trunk diameter.—8.5 cm at 100 cm above ground level.
   Vigor.—Medium.
   Density.—Above average in number of lateral limbs generated by all trees of this variety.
   Form.—Spreading, laterals are moderately upright to about the same degree as ‘Emperor Francis’.
   Production.—Heavy, over 200 pounds per season per tree on Mazzard seedling rootstocks.
   Bearing.—Annual on spurs and on 3 to 4 basal buds of one year old shoots.
   Disease resistance/tolerance.—Similar to ‘Emperor Francis’ in tolerance to brown rot fungus of the blossoms and fruit (rated 7 at Geneva on scale of 1, as worst, to 9, as best); also similar to ‘Emperor Francis’ in tolerance to bacterial canker of the tree and fruit (rated as 7 for tree and 8 for fruit, again with 1, as worst, and 9, as best); highly tolerant of black knot fungus of the wood.
   Cold hardiness.—Very good, crops regularly in test orchards in commercial fruit districts of New York and Michigan where mid-winter temperatures often go below −15 degrees Fahrenheit. Tree livability is very good in commercial sweet cherry orchard districts of the Eastern U.S.A. and is rated by Geneva Experiment Station pomologists as similar to that of its male parent, “Emperor Francis”.
   Frost tolerance.—Very good (rated as 7 at Geneva on scale of 1, as worst, to 9, as best)
   Graft compatibility.—Very good, produces smooth compatible graft unions with Mazzard seedling, Mahaleb seedling, ‘M×M 2’, ‘M×M 60’ and slight overgrowth (as is usually the case for all sweet cherry scion varieties) with ‘Gisela 5’ cherry rootstocks.
Trunk:
   Size.—8.5 cm in diameter at 100 cm above the ground for 6-year-old tree on ‘Gisela 5’ rootstock.
   Surface.—Bark is smooth with prominent, small lenticels.
   Bark color.—Grayed Purple 183 B, where directly exposed to the sun.
   Lenticels.—Grayed Orange 167 B, varying in length from 0.5 to 1.5 cm and width from 0.25 to 0.5 cm, prominent, horizontal, elliptical.
Vegetative buds:
   Placement.—At bud positions 4 and higher number from the base of new growth, and at the tip of each fruit spur.
   Appearance.—Pointed and flatter than flower buds on previous season's annual growth of shoots.
   Color.—Grayed Orangel 65B in fully dormant stage of development.
Leaves:
   Size.—9.0 to 11.5 cm in length, 5.5 to 6.5 cm in width.
   Form.—Symmetrical halves on both sides of central axis.
   Thickness.—Medium.
   Texture.—Crisp, leathery.
   Pubescence.—None on either surface.
   Margin.—Serrations regular and bluntly pointed.
   Adaxial surface.—Green 137A.
   Abaxial surface.—Yellow Green 148C.
   Veination.—Pinnate.
   Vein color.—Adaxial Yellow Green 144D, abaxial Yellow Green 144B.
   Petiole.—6.0 to 6.5 cm length.
   Petiole color.—Red Purple 59A.
   Petiole groove.—Narrow.
   Glands.—1 to 2 on petiole, bluntly globose, length 1.5 to 1.75 mm, width 1 to 1.25 mm, color Orange 28A with occasional glands also present on basal edges of leaf blades.
Flowers:
   Blooming period.—Early, mid-season, April 23 to May 1 in Geneva, N.Y.
   Presentation.—Non-showy, white.
   Fragrance.—Sweet, flowery.
   Fertility.—Self-incompatible, belonging to the pollination compatibility group designated as having S₁S₆ alleles, which are the same as those possessed by ‘Noble’ and ‘NY 518’, hence, making these three varieties incompatible with each other.
   Pollen.—Present, plentiful, good pollen source for pollenizing all other sweet cherries except those with identical S alleles, as stated above.
   Corolla diameter.—32 mm.
   Number of flowers per cluster.—5 to 8.
   Petals.—Single, round, length 10 mm, width 10 mm, 5 in number, slightly overlapping, margin slightly ruffled, soft texture, White 155D.
   Peduncle.—Length 4.8 to 5.2 mm, width 0.4 mm, Green 143B.
   Filament.—Length 8mm, width 0.1 mm, White 155 D.
   Anther.—Profuse pollen, Yellow 13A
   Sepals.—5 in number, Green 143 B, bluntly ovate in shape.
Fruit:
   Maturity when described.—commercial ripeness, 17 degrees brix.
   Date of first picking.—Late, mid-season ripening, July 10 to July 15 at Geneva, N.Y.
   Size.—Weight 8.5 g, length 3.0 cm, width 3.2 cm.
   Form.—Round-oblate appearance.
   Stem cavity.—Wide and tolerant to concentric rain-induced cracking.
   Stem.—length 40 mm, width 1.3 mm, color Green 143 B.
Skin:
   Thickness.—Medium.
   Tendency to crack.—Low to moderate, similar to ‘Emperor Francis, rated as 7 at Geneva (1, worst, to 9 best).
   Color.—Blush on the most intensely pigmented sector of the fruit's skin Red 45C, non-blush area without Anthocyanin pigmentation is Yellow Orange 20B.
   Flavor.—Very good.
Flesh:
   Texture.—Somewhat above average in firmness, rated as 6.5 at Geneva (1, worst, to 9 best).
   Acidity.—Medium until sugar levels get to above mid-teen levels in degrees brix.
   Flavor.—Very good.
   Aroma.—Present, pleasing.
   Color.—Yellow Orange 14C.
   Coloration in the pit cavity.—Same as flesh.
   Eating quality.—Very good when mature to above mid-teen levels of degrees brix and excellent when eaten at high-teen levels of degrees brix.
Stone:
   Size.—1 cm in length, 1 cm in width at the widest point near the center, 1.1 cm in width at the widest point of the flatter direction.
   Sides.—Ridges, 3 to 4 in number, with varying amounts of encircling of the suture side of the pit.
   Type.—Semi-freestone a commercial maturity.
   Tendency to crack.—Non-existent in nature.
Use: Processing for production of maraschino-type and glass-type fruit for uses in the liquor, bakery and dairy industries has been highly successful in pilot trials. Machine harvest through use of trunk shakers has also been highly successful in pilot trials. It is well suited to commercial grower's needs in that it possesses heavy yielding characteristics under relatively adverse climatic conditions found in processing sweet cherry districts of the Eastern U.S.A. This variety will be highly sought after by processing cherry growers in the Eastern U.S.A. to be used as a highly effective pollinator for commercial, processing orchards of ‘Emperor Francis’ and ‘Ulster’ and because its fruit ripen mid-way between ‘Emperor Francis’ and ‘Starks Gold’, the two most widely planted, processing sweet cherry varieties grown in the Eastern U.S.A. for maraschino-type cherry manufacturing.

1. ‘Pendleton’, a new and distinct variety of sweet cherry tree, Prunus avium, substantially as herein shown and described, characterized as to novelty by the unique combination of hardy, highly productive tree with successful pollination compatibility with all varieties except those having the S₁S₆ alleles.
